(19) United States
(12) Patent Application Publication
(10) Pub. No.: US 2008/0317624 A1
(43) Pub. Date: Dec. 25, 2008

(54) METHOD OF DISINFECTING CONTAINERS WITH A DISINFECTANT AND PRIOR HEAT TREATMENT, AND A CORRESPONDING INSTALLATION

(75) Inventors: Delphine GUEGUEN, La Ferte Bernard (FR); Guy Dumargue, Cherre (FR)

Correspondence Address: Muncy, Geissler, Olds & Lowe, PLLC, P.O. BOX 1364, FAIRFAX, VA, 22038-1364, US

(73) Assignee: SERAC GROUP

(21) Appl. No.: 12/141,579

(22) Filed: Jun. 18, 2008

(30) Foreign Application Priority Data
Jun. 19, 2007 (FR) 07 04351

(51) Int. Cl. A61L 2/07; A61L 2/18

(52) U.S. Cl. 422/26; 422/28

(57) ABSTRACT

The method of aseptically treating containers in accordance with the invention comprises the step of subjecting the containers while held upside-down in a heat treatment station to steam at 140° C. for 2 s to 20 s and then in subjecting the containers in a station to a disinfectant mixture comprising peracetic acid, hydrogen peroxide, acetic acid, and water raised to a maximum temperature of 60° C.

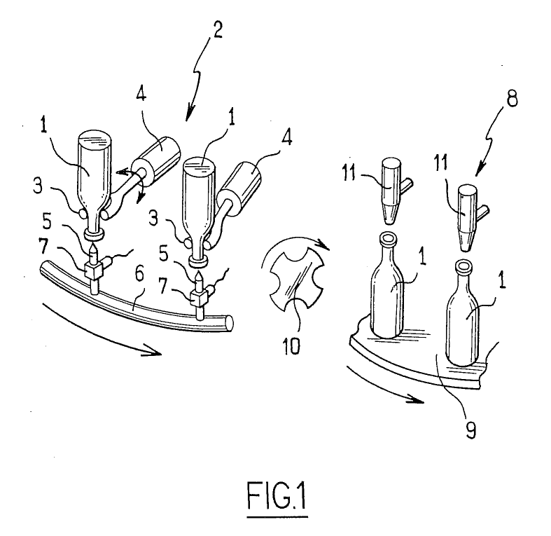

### BACKGROUND OF THE INVENTION

It is known that in order to ensure that a substance is packaged in containers under good aseptic conditions, it is necessary for the containers themselves to be subjected to disinfection treatment upstream from the filler installation.

Disinfection treatment consists in treating containers with a disinfectant, for example a mixture of peracetic acid, hydrogen peroxide, acetic acid, and water. In known methods, the mixture generally has a concentration of 1800 parts per million (ppm) of peracetic acid, with the containers being filled with a mixture at 50° C. and the mixture being maintained in the containers for 20 seconds (s).

During the treatment, some of the peracetic acid transforms into acetic acid, water, and oxygen. In existing methods, the mixture is recycled with added solution for returning the mixture to the desired concentration. This avoids discarding the remainder of the mixture.

It has nevertheless been found that the present method does not enable certain particularly-resistant pathogenic microorganisms to be destroyed, in particular bacteria known as Bacillus cerus. In order to destroy those pathogenic microorganisms, proposals have been made to increase the concentration of the mixture to 4000 ppm and to treat containers with a mixture at 65° C. for 20 s. Unfortunately, the aggressivity of acetic acid increases with temperature, such that treatment under the conditions proposed leads to a significant increase in the corrosion of the machinery and runs the risk of leading rapidly to deterioration thereof. Furthermore, the instability of peracetic acid increases with temperature, such that a larger fraction of the peracetic acid contained in the mixture decomposes during the treatment at 65° C., and it becomes difficult to reuse the remaining mixture for recycling after restoring the concentration to the appropriate level. The remaining mixture is thus discarded to the drains, resulting in environmental consequences and also in increased cost of treatment, since peracetic acid is particularly expensive.

It has also been envisaged to maintain the treatment under present conditions of concentration and temperature while lengthening treatment time. Nevertheless, the disinfection treatment is generally performed by filling the containers with the disinfectant. It is therefore necessary to store the containers while full in storage areas of volume that becomes prohibitive.

### OBJECT OF THE INVENTION

The object of the invention is to propose a method and an installation for disinfectant treatment that enables resistant pathogens to be destroyed, while minimizing side effects.

### SUMMARY OF THE INVENTION

In order to achieve this object, the invention provides a method of disinfecting containers, the method comprising the steps of subjecting the containers to a disinfectant mixture comprising peracetic acid, hydrogen peroxide, acetic acid, and water, and in activating the mixture by heat treatment to a temperature of at least 60° C., which activation is performed in such a manner that the disinfectant mixture does not exceed a temperature of 60° C. The heat treatment is preferably performed prior to treating the containers with the disinfectant mixture.

It has been found that the heat treatment enables the mixture to be raised to or maintained at a degree of activation that enables pathogens to be destroyed better, while applying the disinfectant mixture under conditions that are identical or analogous to those in the prior art treatment.

### BRIEF DESCRIPTION OF THE DRAWING

Other characteristics and advantages of the invention appear on reading the following description of a preferred embodiment of the invention, given with, reference to the sole accompanying FIGURE, which is a fragmentary diagrammatic view of a disinfecting installation including a heat treatment station and an associated station for treatment with a disinfectant.

### DETAILED DESCRIPTION OF THE INVENTION

With reference to the figure, the method of the invention is advantageously implemented by initially introducing containers 1 into a disinfecting installation comprising a heat treatment station having container supports such as forks 3 suitably connected to control motors 4 for causing the containers 1 to pivot between an upright position (not shown in the figure) and an upside-down position, as shown in the figure, in which the containers 1 are brought over nozzles 5 for delivering steam, each nozzle 5 being connected to a feed manifold 6 via a controlled valve 7.

In a preferred implementation of the invention, the method comprises a heat treatment step prior to a step of treatment by means of a disinfectant mixture that comprises peracetic acid, hydrogen peroxide, acetic acid, and water. The heat treatment is performed by projecting steam at 140° C. into the insides of the containers 1 for two seconds while the containers are held upside-down.

This position for the containers enables the water that condenses on the container wall to be eliminated so that the container continues to be subjected to steam at 140° C. throughout the duration of the treatment. With reference to a travel direction of the containers 1 through the installation, the heat treatment station is disposed immediately upstream from a station 8 for treatment with the disinfectant mixture, the disinfectant treatment station comprising in conventional manner a rotary platform 9 onto which the containers 1 are transferred by means of a transfer star wheel 10, each container 1 then being placed beneath a filler spout 11 that is suitably fed with disinfectant mixture. The filled containers 1 are then transferred in known manner (not shown) to a storage zone from which they are taken for emptying and then rinsing.

The step of treatment with the disinfectant mixture can then be maintained under the usual conditions of concentration, temperature, and duration to be found in existing methods, with some of the parameters possibly being reduced, e.g. the duration of treatment, thus enabling space to be saved, thereby compensating for the space occupied by the heat treatment installation.

In any event, the temperature and the duration of heat treatment are calculated so that the disinfectant mixture is raised to a maximum temperature equal to 60° C. in order to minimize decomposition of peracetic acid, taking account of all of the parameters, and in particular the thermal inertia of the containers.

Naturally, the invention is not limited to the preferred implementation described above. It has found that similar effects are obtained with heat treatment using water or steam at a temperature lying in the range 60° C. to 140° C. for a duration lying in the range 2 s to 20 s.

The heat treatment may also be performed by other means, in particular with a hot gas, the object of the treatment being to bring or maintain the disinfectant mixture in a state of activation that is sufficient for destroying the pathogens but without causing unwanted decomposition of the components of the disinfectant.

Although the heat treatment and the treatment by means of the disinfectant mixture in the implementation shown are represented solely as involving treatment of the inside faces of the containers, it is also possible to perform the treatment and disinfectant treatment on the outside faces of the containers by successively spraying water or steam and then disinfectant.

Although the method of the invention is described above with the heat treatment step being performed prior to applying the disinfectant mixture, thus making it possible to have good control over the temperature of the wall of the container, it is also possible to perform the heat treatment after the disinfectant mixture has been applied.

What is claimed is:

1. A method of disinfecting containers, the method including a step of treating the containers with a disinfectant mixture comprising peracetic acid, hydrogen peroxide, acetic acid, and water, the method further comprising a step of subjecting the containers to heat treatment at a temperature of at least 60° C. so that the disinfectant mixture is raised to a temperature no greater than 60° C.

2. An aseptic treatment method according to claim 1, wherein the heat treatment is performed before the treatment with the disinfectant mixture.

3. An aseptic treatment method according to claim 1, wherein the heat treatment is performed with hot water or with steam.

4. An aseptic treatment method according to claim 3, wherein the heat treatment is implemented while the containers are held upside-down.

5. An aseptic treatment method according to claim 1, wherein the heat treatment is performed for a duration lying in the range 2 s to 20 s.

6. An aseptic treatment method according to claim 3, wherein the heat treatment is performed with steam at 140° C.
